(19) United States
(12) Plant Patent Application Publication
(10) Pub. No.: US 2025/0048946 P1
(43) Pub. Date: Feb. 6, 2025

(54) Olive tree named 'OAC9804-07'

(50) ;

(71) Applicant: Agromillora Catalana S.A.U., Sant Sadurni d'Anoia (ES)

(72) Inventors: Salvatore Camposeo, Valenzano (IT); Gaetano Alessandro Vivaldi, Barletta (IT); Marisa Cunill, Barcelona (ES)

(73) Assignee: Agromillora Catalana S.A.U., Sant Sadurni d'Anoia (ES)

(21) Appl. No.: 18/230,006

(22) Filed: Aug. 3, 2023

(51) Int. Cl. A01H 6/00; A01H 5/08

(52) U.S. Cl. CPC A01H 6/00; A01H 5/08
USPC PLT/158

(57) ABSTRACT

A new distinct cultivar of Olea europaea named ‘OAC9804-07’ is described. characterized by a spreading tree habit, black fruit color, and suitability for both table olive and oil production. The new variety is an olive tree, suitable for high density olive orchards.

Latin name of the genus and species: Olea europaea.

Variety denomination: ‘OAC9804-07’.

### BACKGROUND OF THE INVENTION

The present invention is a new and distinct variety of olive tree named ‘OAC9804-07’. ‘OAC9804-07’ is the product of a planned breeding program by the inventor in Barcelona, Spain. The new cultivar is the result of a cross between the seed parent, Olea europaea ‘Arbosana’, and the pollen parent, Olea europaea ‘Blanqueta de Elbas’, unpatented.

‘OAC9804-07’ was selected as an interesting new variety in 2016. Asexual reproduction by way of rooting semi-ligneous stem cuttings was first performed during 2016, in Barcelona, Spain. Through 14 subsequent generations, the unique features of ‘OAC9804-07’ have proven to be stable and true to type.

### SUMMARY OF THE INVENTION

The cultivar ‘OAC9804-07’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘OAC9804-07’ as a new and distinct Olea europaea cultivar:
1. Upright to spreading habit.
2. Black fruit color.
3. High fertility.
4. High frequency of mixed shoots that produce vegetative buds and flower buds.
5. Suitability for use as both table and oil olives.
6. Resistance to leafspot (Spilocaea oleagina).

### COMPARISONS TO PARENT VARIETIES

Plants of the new cultivar ‘OAC9804-07’ are similar to plants of the seed parent in most horticultural characteristics, however, plants of the new variety differ in the following:
1. The new variety produces fruit having a medium weight range; fruit weight of the seed parent variety is low.
2. Vigor of the new variety is medium to low; vigor of the seed parent is low.

Plants of the new cultivar ‘OAC9804-07’ are similar to plants of the pollen parent in most horticultural characteristics, however, plants of the new variety differ in the following:
1. The new variety produces larger fruit than the pollen parent.
2. The new variety has exhibited resistance to leafspot, which the pollen parent has not.

### COMMERCIAL COMPARISON

Plants of the new cultivar ‘OAC9804-07’ can be compared to Olea ‘Arbosana’, unpatented. The varieties are similar in most horticultural characteristics, however, the new variety differs in the following:
1. The new variety produces fruit having a medium weight range; fruit of this comparator is low.
2. Vigor of the new variety is medium to low; vigor of this comparator is low.

### BRIEF DESCRIPTION OF THE PHOTOGRAPHS

The accompanying photographic illustrations show typical samples of fruit on 4-year-old specimens of the new variety, in color as nearly true as it is reasonably possible to make in a color illustration of this character. Plants were grown in Barcelona, Spain. Colors in the photograph may differ from the color values cited in the detailed botanical description below, which accurately describes the colors of the new variety.

FIG. 1 illustrates ripe fruit, flesh and stones of ‘OAC9804-07’.

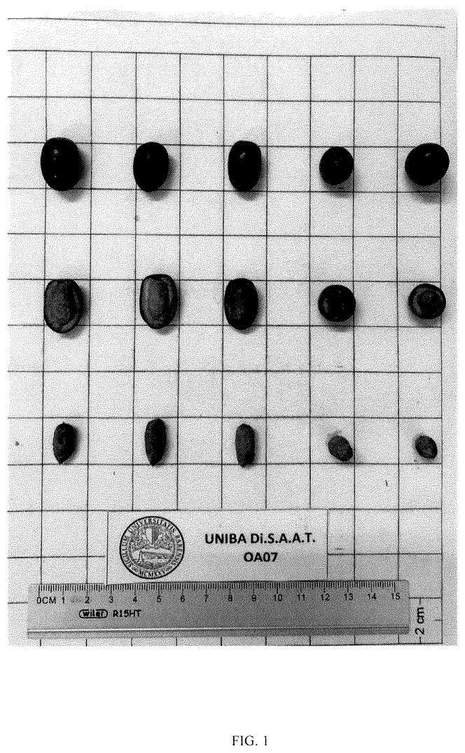

FIG. 2 illustrates ripening fruits of ‘OAC9804-07’ on the tree.

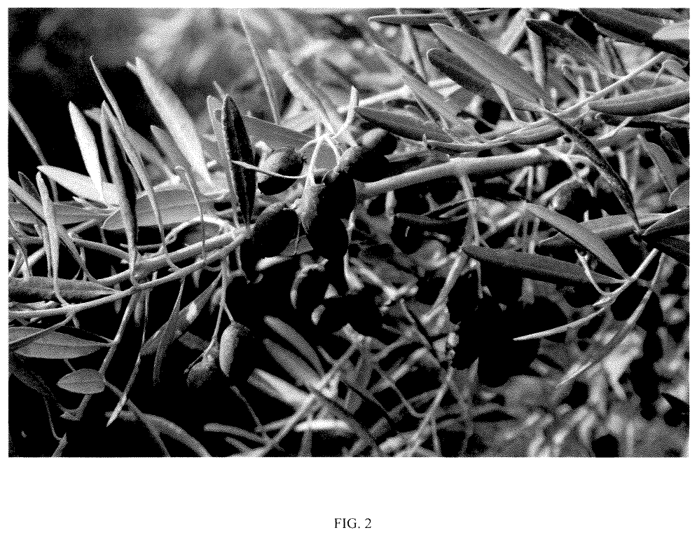

FIG. 3 illustrates unripe fruit of ‘OAC9804-07’.

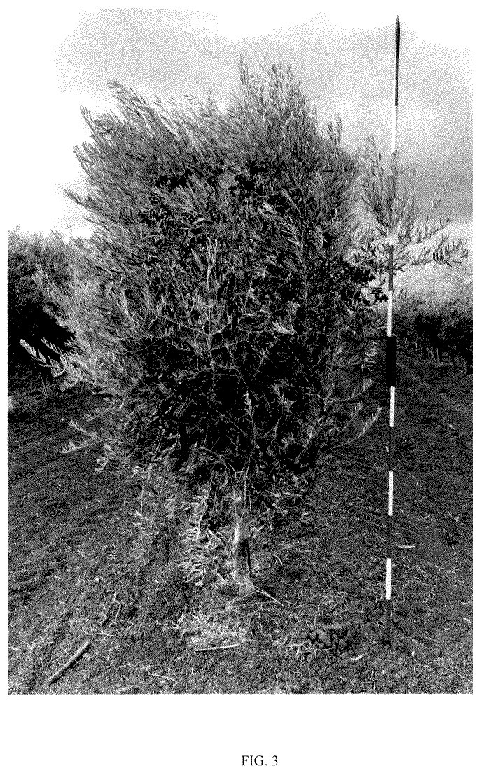

### DETAILED BOTANICAL DESCRIPTION

In the following description, color references are made to The Royal Horticultural Society Colour Chart 2007, except where general terms of ordinary dictionary significance are used. The following observations and measurements describe ‘OAC9804-07’ plants grown outdoors in Barcelona, Spain. The age of plant described is of 4 years, with an average height of 200 cm, a high canopy density, a 300 cm plant spread, a one-year growth rate to reach two meters high and 20 to 30 days to initiate roots at approx. 20° C. At observation location temperature ranged between 15° C. to 35° C. during the day, and between 5° C. to 20° C. during the night.
Botanical classification: Olea europaea ‘OAC9804-07’.

### PROPAGATION

Propagation method: Semi-ligneous stem cuttings.

### TREE

Age of the tree described: About 4 years.
Growth habit: Spreading.
Plant vigor: Moderate to low.

### FOLIAGE

Leaf:
   Arrangement.—Opposite.
   Average length.—4 to 6 cm.
   Average width.—About 1 cm.
   Shape of blade.—Elliptic.
   Apex.—Acute.
   Base.—Tapered.
   Attachment.—Petiolate.
   Margin.—Entire.
   Texture of top surface.—Glabrous.
   Texture of underside.—Very slightly pubescent.
   Aspect.—Flat with slight cupping under near margin.
   Color.—Mature foliage upper side: Near Green 143A. Mature foliage under side: Near Green 138B.
   Petiole.—Length: About 3 to 5 mm. Width: About 2 mm. Pubescence: Glabrous. Color: Near Green 138C.

### FLOWER

Inflorescence and flower type and habit: Panicle composed of perfect, simple cruciform, inconspicuous flowers.
Flower size:
   Diameter.—About 4 mm.
   Depth.—About 2 mm.
Petals:
   Arrangement.—Cruciform.
   Petal quantity.—4.
   Length.—About 3 mm.
   Width.—About 2 mm.
   Texture.—Glabrous.
   Apex.—Acute.
   Base.—Fused.
   Margin.—Entire.
   Color.—Near White 155A.
Calyx/sepals:
   Quantity per flower.—4.
   Arrangement.—Rotate.
   Shape.—Irregular orbicular.
   Length.—About 1 mm.
   Width.—About 1 mm.
   Texture.—Glabrous.
   Color.—Near Yellow-Green 145D.

### REPRODUCTIVE ORGANS

Stamens:
   Number.—2.
   Filament length.—About 0.5 mm.
Anthers:
   Shape.—Hemispherical.
   Length.—1 mm.
   Color.—Near Yellow-Green 145C.
   Pollen.—Color: Near Yellow 4C. Quantity:
Pistil:
   Number.—1.
   Length.—About 1 mm.
   Style.—Length: About 0.5 mm. Color: Near Yellow 4D.
   Stigma.—Shape: Conical, bifid. Color: Near Yellow 4C.

### FRUIT

Weight: Average 2.69 grams.
Color: RHS Black 202A when fully ripe.
Size: Average length: 20.12 mm.
   Average diameter.—7.70 mm.
Symmetry: Weakly asymmetric.
Shape: Rounded.
Nipple: Absent or weak.
Stone shape: Moderately elongated.
Stone weight: High. Average 0.70 grams.
Stone symmetry: Weakly asymmetric.
Stone mucron: Present.
Stone rugosity: Medium.

### OTHER CHARACTERISTICS

Disease/pest resistance: Neither resistance nor susceptibility to normal disease and pests of Olea have been observed.
Temperature tolerance: Tolerant to USDA hardiness zones 8 through 10; exhibits good resistance to low temperatures.

What is claimed is:

1. A new and distinct cultivar of Olive tree named ‘OAC9804-07’ as herein illustrated and described.
